(19) United States
(12) Patent Application Publication
(10) Pub. No.: US 2006/0140921 A1
(43) Pub. Date: Jun. 29, 2006

(54) Disimmortalizable mammalian chromaffin cell lines for cell therapy for pain

(76) Inventors: Mary J. Eaton, Miami Beach, FL (US); Yves Lazorthes, Toulouse (FR); Jean-Paul Herman, Aix-En-Provence (FR)

Correspondence Address: DINESH AGARWAL, P.C., 5350 SHAWNEE ROAD, SUITE 330, ALEXANDRIA, VA, 22312, US

(21) Appl. No.: 11/253,731

(22) Filed: Oct. 20, 2005

Related U.S. Application Data
(60) Provisional application No. 60/620,660, filed on Oct. 22, 2004.

(51) Int. Cl. A61K 48/00; C12N 5/08

(52) U.S. Cl. 424/93.21; 435/366

(57) ABSTRACT

Cloned disimmortalizable mammalian chromaffin cell lines that express chromaffin cell phenotype/genotype are disclosed.

### CROSS-REFERENCE TO RELATED APPLICATIONS

The present application claims priority on prior U.S. Provisional Application Ser. No. 60/620,660, filed Oct. 22, 2004, which is hereby incorporated herein in its entirety by reference.

### STATEMENT REGARDING FEDERALLY SPONSORED RESEARCH OR DEVELOPMENT

The work leading to the present invention was supported by one or more grants from the U.S. Government, including Veterans Affairs Merit Grants. The U.S. Government therefore has certain rights in the invention.

### FIELD AND BACKGROUND OF THE INVENTION

The present invention is generally directed to the treatment of neuropathic pain associated with cancer, nerve injury, and/or spinal cord injury. More particularly, the present invention is directed to the development of mammalian chromaffin cell lines for diagnostic, therapeutic, and/or other purposes.

Despite recent improvements in pain relief, many patients continue to suffer from intractable pain. Cellular therapy, such as intrathecal (I-Th) transplant of primary adrenal chromaffin cells (CC), resulted in analgesic effects in various pain models (References 6 and 22). Such a clinical protocol at large-scale level is limited by low availability of donor tissue (References 7 and 14). An improved CC therapy approach has been proposed, with CC lines conditionally immortalized with the temperature-sensitive allele of SV40 Tag (tsTag) to turn off the immortalizing agent (References 5 and 10). Such a strategy requires a change in temperature in the transplant situation to downregulate a functional oncogene, and these cells have the problem of reduced catecholamine and opioid peptide synthesis, due to incomplete extinction of the tsTag (Reference 10). Reversible immortalization using site-specific DNA recombination and cre/lox excision of the Tag sequence (or any immortalizing oncogenic sequences, such as v-myc) should be a safer strategy for clinical applications, since the oncogene is completely removed before transplant and the phenotype of disimmortalized cells is more normal (References 9 and 16). Rat CC immortalized with the loxP flanked tsTag was reported using modulatable Cre recombinase activity that can be activated by the synthetic steroid RU486 (Reference 12). As such conditionally immortalized CC are stable and appear homogeneous (Reference 10), they could be an ideal candidate for further genetic manipulation, as a model for the creation of human chromaffin cell lines.

The initial technology for lumbar spinal transplants of adrenal chromaffin tissues from murine, bovine and human sources has been developed and shown to be effective in animal models of neuropathic pain and, more recently, in clinical trials for the management of human cancer pain. Such primary tissue sources are not, however, homogeneous since the need for an adequate number of cells requires that they be obtained from multiple donors. A significant advance in this technology and in the ability to manipulate, through bio-engineering, the survival and output of neuroactive substances from these adrenal chromaffin cells for pain would be the creation and development of immortalized chromaffin cells. Such cells would provide an unlimited, homogenous source of cells for transplant and in vitro manipulation and evaluation.

The disimmortalizable human chromaffin cell lines, able to be made completely safe for transplant in humans, which also reverse or reduce tonic and neuropathic pain after transplant into the subarachnoid space near the spinal cord, will provide a stable, well-characterized source of antinociceptive chromaffin tissue which can be further genetically modified, such as by the addition of opioid genes for overexpression of enkephalins, endomorphins, or chromagranins, to boost their antinociceptive potential. These human chromaffin cell lines can be developed for clinical use to treat the devastating problems of neuropathic pain associated with cancer, nerve injury and spinal cord injury.

While the use of such immortalized chromaffin cells for transplant would be advantageous for a number of reasons, the presence of a potentially dangerous oncogenic sequence that is used for immortalization in the cells would preclude their use in humans unless one could inactivate or remove the immortalizing construct from the cells before transplant. The ability to remove a potentially dangerous sequence from a useful cell line will be applicable to the eventual use of these disimmortalizable chromaffin cells for chronic pain in humans. We have modeled this strategy and used molecular methods for a modulatable cre/lox site-directed excision of the oncogenic sequence before cell transplant in animal models as a pre-clinical test of this strategy for use of chromaffin cell lines for pain control. Such a strategy has been previously modeled in the creation and use of disimmortalizable rat chromaffin cell lines for the reduction of neuropathic pain (References 12 and 23).

### OBJECTS AND SUMMARY OF THE INVENTION

The principal object of, the present invention is to provide a disimmortalizable mammalian chromaffin cell line having diagnostic, therapeutic, and/or other utilities. in particular, the cell line would have utility in cell therapy for pain.

An object of the present invention is to provide unlimited homogeneous chromaffin cells for transplant, in vitro manipulation, and/or evaluation.

Another object of the present invention is to provide a disimmortalizable mammalian cell line which is safe for transplant in humans, reverses or reduces tonic and/or neuropathic pain after transplant into the subarachnoid space near the spinal cord.

Yet another object of the present invention is to provide a disimmortalizable mammalian cell line which is a well-characterized source of antinociceptive chromaffin tissue that can be further genetically modified or manipulated, such as by the addition of opioid genes for overexpression of enkephalins, endomorphins, and/or chromagranins, to boost their antinociceptive potential.

An additional object of the present invention is to provide a disimmortalizable mammalian cell line which can be developed for clinical use to treat neuropathic pain normally associated with cancer, nerve inquiry, and/or spinal cord inquiry.

A further object of the present invention is to provide an immortalized human chromaffin cell line.

Another object of the present invention is to provide a cloned chromaffin cell line which expresses chromaffin cell phenotype.

Yet another object of the present invention is to provide a cloned human chromaffin cell line which expresses chromaffin cell genotype.

Still yet another object of the present invention is to provide a cloned human chromaffin cell line which synthesizes and releases chromaffin-related antinociceptive agents, such as adrenaline, noradrenaline, and met-enkephalin.

Still yet another object of the present invention is to provide a cloned human chromaffin cell line which can be disimmortalized.

In summary, we have immortalized embryonic human chromaffin tissue derived from human fetal tissue from gestational age of 11-14 weeks by infection with an amphotropic virus encoding the sequence for v-myc flanked by loxP sites. This oncogenic construct also encodes a positive/negative antibiotic cassette expressing neomycin and tyrosine kinase for both positive and negative selection of immortalized chromaffin cells. We developed an in situ hybridization method, utilizing a riboprobe for ‘neomycin’, a part of the sequence expressed by the immortalized cells, as a way to mark both the immortalized cells and to later demonstrate the disappearance of the v-myc oncogene with RU486/gancyclovir treatment for disimmortalizationn before transplant. We plan to (1) transfect these disimmortalizable human chromaffin cells with an amphotropic virus encoding the sequence for a steroid-binding fusion-protein that includes the Cre protein and the mutant steroid-binding receptor, which responds to RU486 in vitro; and (2) characterize cloned human chromaffin cell lines for: (a) expression of the chromaffin cell phenotype with markers for the catecholamine-synthesizing enzymes; (b) synthesis and release of chromaffin-related antinociceptive agents such as adrenaline, noradrenaline and met-enkephalin; and (c) the ability to be efficiently disimmortalized with RU486 and gancyclovir before transplant in vitro. Potential disimmortalizable human chromaffin cell lines will be tested for their antinociceptive potential in a variety of tonic and chronic pain animal models including formalin hindpaw injection, partial nerve injury, and excitotoxic spinal cord injury pain and other SCI animal models.

One of the above objects is met, in part, by the present invention which in one aspect includes an immortalized mammalian cell that expresses chromaffin cell phenotype.

Another aspect of the present invention includes an immortalized mammalian cell that expresses chromaffin cell genotype.

Another aspect of the present invention includes a cloned mammalian cell that expresses chromaffin cell phenotype.

Another aspect of the present invention includes a cloned mammalian cell that expresses chromaffin cell genotype.

Another aspect of the present invention includes a disimmortalizable mammalian cell that expresses chromaffin cell phenotype.

Another aspect of the present invention includes a disimmortalizable mammalian cell that expresses chromaffin cell genotype.

Another aspect of the present invention includes a cloned mammalian cell line that releases a chromaffin-related antinociceptive agent.

Another aspect of the present invention includes a disimmortalizable mammalian chromaffin cell line.

Another aspect of the present invention includes a disimmortalizable human chromaffin cell line.

Another aspect of the present invention includes a disimmortalizable mammalian chromaffin cell line for transplant into a region of the central nervous system of a subject to induce analgesia.

Another aspect of the present invention includes a disimmortalizable human chromaffin cell line for transplant into a region of the central nervous system of a subject to induce analgesia.

### BRIEF DESCRIPTION OF THE DRAWINGS

One of the above and other objects, novel features and advantages of the present invention will become apparent from the following detailed description of the preferred embodiment(s) of the invention, as illustrated in the drawings, in which:

FIG. 1 is a phase contrast micrograph of human chromaffin cells immortalized with pMycTN (loxP). Clusters of immortalized human chromaffin cells (arrows) are disclosed on a bed of immortalized human adrenal fibroblasts. Such cells likely require the fibroblast feeder to survive and divide in vitro, but can easily be separated from these non-chromaffin cells by differential plating methods.

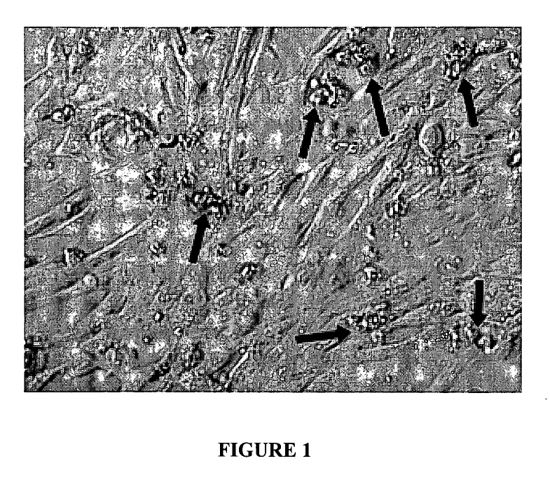

FIG. 2 illustrates the chromaffin phenotype and PNMT in immortalized human chromaffin cells. Cultures of human chromaffin cells immortalized with pMycTN (loxP), were stained with an antibody directed against phenylethanolamine-N-methyltransferase (PNMT), which indicates a chromaffin cell phenotype (bright cells, arrows). The fibroblast feeder layer does not stain for PNMT. Human chromaffin cells (hCCs) are round, unlike the spindle shaped fibroblasts.

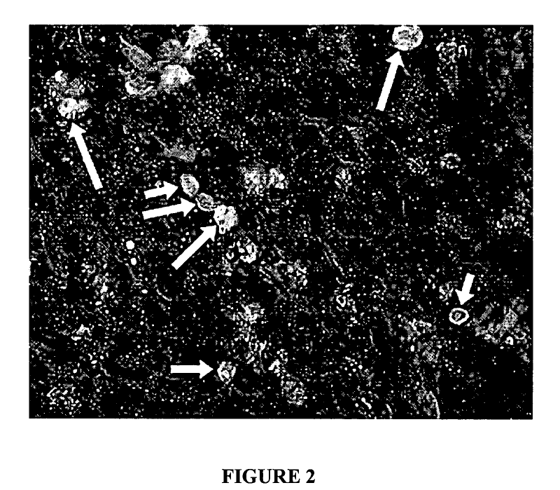

FIG. 3A is a low magnification micrograph of immortalized hCCs after dig-insitu hybridization for an antisense riboprobe directed against the neomycin sequence. Cultures of hCCs immortalized with pMycTN (loxP) were hybridized with an antisense digoxigenin-labeled riboprobe directed against the neomycin sequence. This neomycin sequence is part of the pMycTN (loxP) vector encoded by the immortalized hCCs (after selection with G418 antibiotic). At low magnification, the hCCs are only labeled with the antisense probe (arrows), suggesting that under the same hybridization conditions, the antisense neo probe recognizes the complementary neo sequence in the cells and will be useful to monitor the disimmortalization completeness with activation of CrePR1 in the cells.

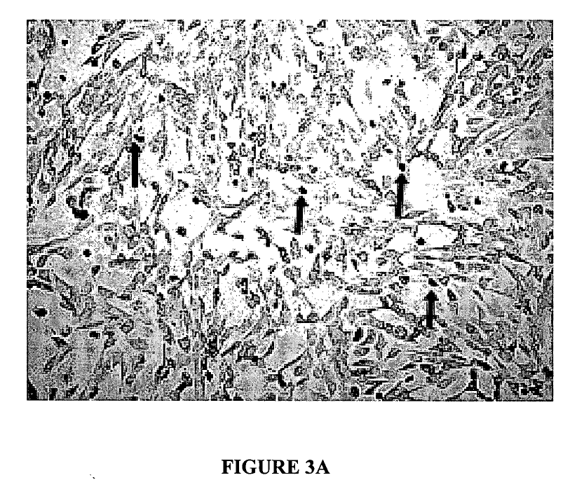

FIG. 3B is a low magnification micrograph of immortalized hCCs after dig-insitu hybridization for a sense riboprobe directed against the neomycin sequence. Cultures of hCCs immortalized with pMycTN (loxP) were hybridized with a sense digoxigenin-labeled riboprobe direct against the neomycin sequence. This neomycin sequence is part of the pMycTN (loxP) vector encoded by the immortalized hCCs (after selection with G418 antibiotic). At low magnification, the hCCs are only labeled with the antisense probe not the sense probe used above, suggesting that under the same hybridization conditions, the antisense neo probe recognizes the complementary neo sequence in the cells and will be useful to monitor the disimmortalization completeness with activation of CrePR1 in the cells.

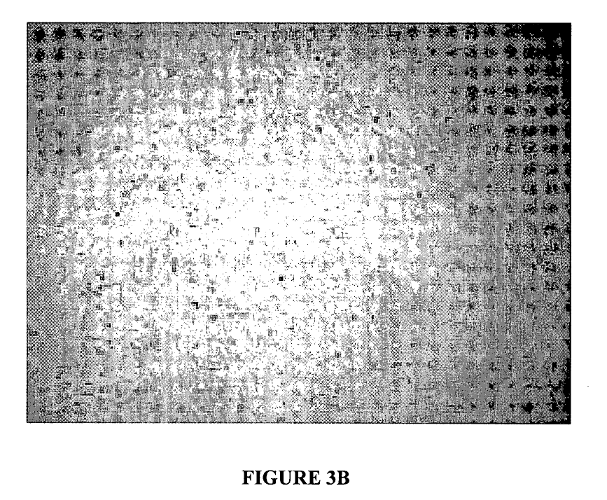

FIG. 4A is a high magnification micrograph of immortalized hCCs after dig-insitu hybridization for an antisense riboprobe directed against the neomycin sequence. Cultures of hCCs immortalized with pMycTN (loxP) were hybridized with an antisense digoxigenin-labeled riboprobe direct against the neomycin sequence. This neomycin sequence is part of the pMycTN (loxP) vector encoded by the immortalized hCCs (after selection with G418 antibiotic). At low magnification, the hCCs are only labeled with the antisense probe (arrows), suggesting that under the same hybridization conditions, the antisense neo probe recognizes the complementary neo sequence in the cells and will be useful to monitor the disimmortalization completeness with activation of CrePR1 in the cells.

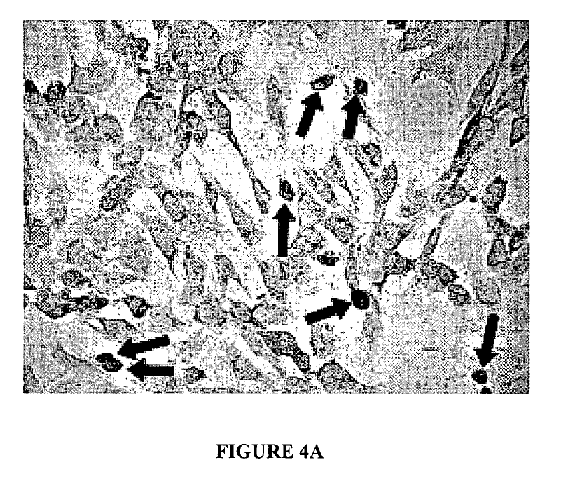

FIG. 4B is a high magnification micrograph of immortalized hCCs after dig-insitu hybridization for a sense riboprobe directed against the neomycin sequence. Cultures of hCCs immortalized with pMycTN (loxP) were hybridized with a sense digoxigenin-labeled riboprobe direct against the neomycin sequence. This neomycin sequence is part of the pMycTN (loxP) vector encoded by the immortalized hCCs (after selection with G418 antibiotic). At low magnification, the hCCs are only labeled with the antisense probe not the sense probe used above, suggesting that under the same hybridization conditions, the antisense neo probe recognizes the complementary neo sequence in the cells and will be useful to monitor the disimmortalization completeness with activation of CrePR1 in the cells.

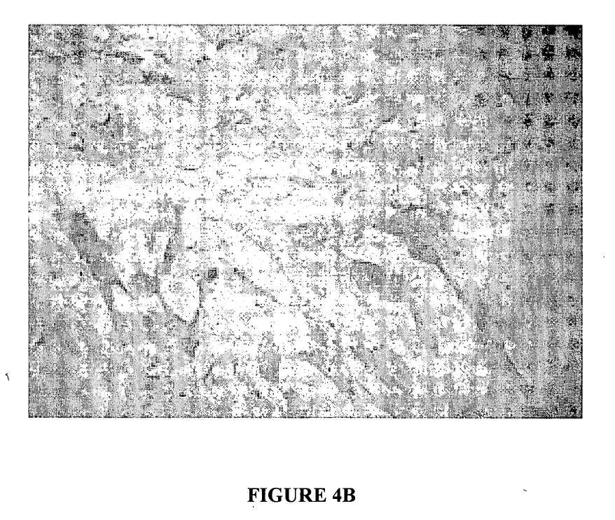

FIG. 5 is a phase contrast micrograph of human chromaffin cells immortalized with pTTN (loxP), the wild-type SV40 large T-antigen and p1710 (CrePR1), for later disimmortalization. The cells had been expanded at 37° C. followed by differential plating, to remove accompanying adrenal fibroblasts.

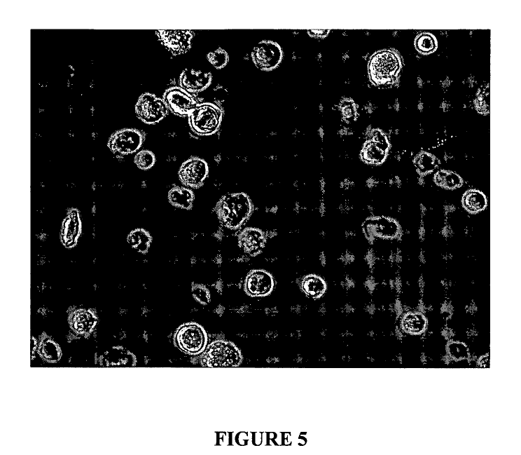

FIG. 6A illustrates the chromaffin phenotype and tyrosine hydoxylase (TH) in immortalized human chromaffin cells after differential plating. Cultures of human chromaffin cells immortalized with TTN/CrePR1 were stained with an antibody directed against tyrosine hydoxylase (TH), which indicates a catecholamine chromaffin cell phenotype, since TH is the rate-limiting enzyme for catecholamine synthesis in primary chromaffin cells after differential plating. The TH immunohistochemistry signal is low in immortalized human chromaffin cells.

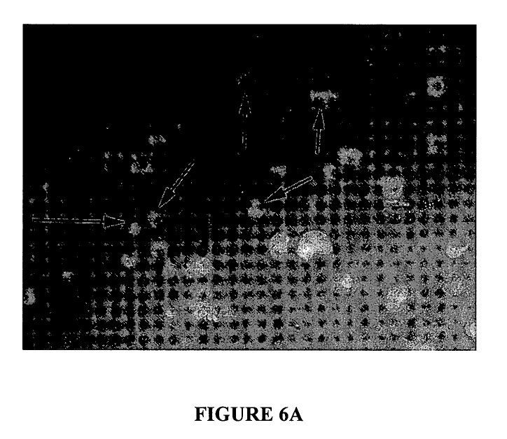

FIG. 6B illustrates the chromaffin phenotype and dopa beta hydroxylase (DβH) in immortalized human chromaffin cells (arrows) after differential plating. Cultures of human chromaffin cells immortalized with TTN/CrePR1 were stained with an antibody directed against dopa beta hydroxylase (DβH), which indicates a catecholamine chromaffin phenotype, since DβH is the enzyme that aids in the conversion of dopamine to norepinephrine in primary chromaffin cells. The DβH immunohistochemistry signal is moderate in immortalized human chromaffin cells.

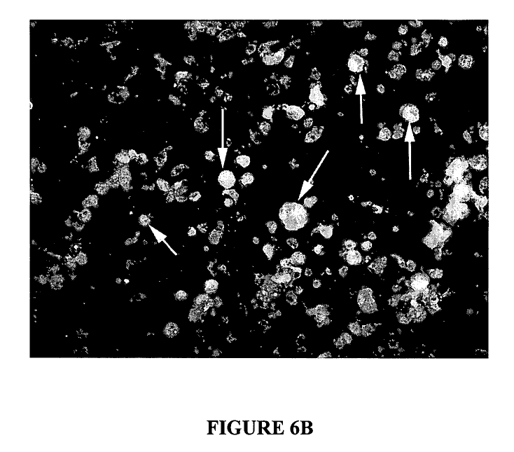

FIG. 6C illustrates the chromaffin phenotype and phenylethanolamine-N-methyltransferase (PNMT) in immortalized human chromaffin cells (arrows) after differential plating. Cultures of human chromaffin cells immortalized with TTN/CrePR1 were stained with an antibody directed against PNMT, which indicates a catecholamine chromaffin phenotype, since DβH is the enzyme that aids in the conversion of norepinephrine to epinephrine in primary chromaffin cells. The PNMT immunohistochemistry signal is strong in immortalized human chromaffin cells.

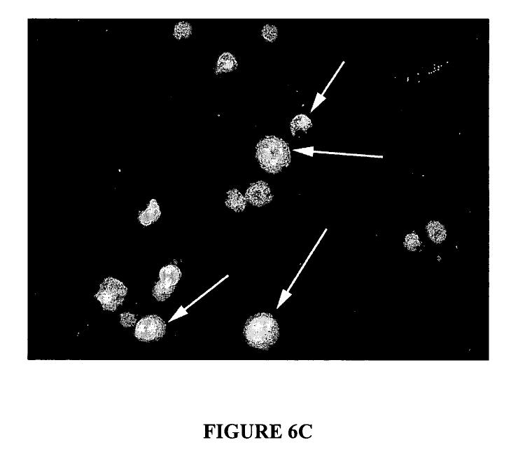

FIG. 7A illustrates the chromaffin phenotype and met-enkephalin (MET-ENK) in immortalized human chromaffin cells (arrows) after differential plating. Cultures of human chromaffin cells immortalized with TTN/CrePR1 were stained with an antibody directed against MET-ENK, which indicates an opioid chromaffin phenotype in immortalized human chromaffin cells. The MET-ENK immunohistochemistry signal is moderate in immortalized human chromaffin cells.

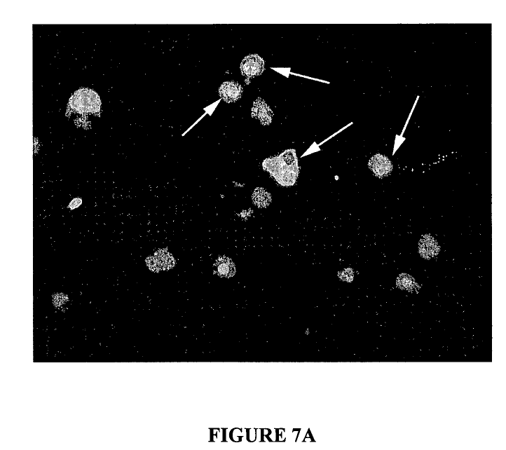

FIG. 7B illustrates the chromaffin phenotype and the peptide chromagranin in immortalized human chromaffin cells (arrows) after differential plating. Cultures of human chromaffin cells immortalized with TTN/CrePR1 were stained with an antibody directed against chromagranin, which indicates a chromagranin chromaffin phenotype in immortalized human chromaffin cells. The chromagranin immunohistochemistry signal is moderate to high in immortalized human chromaffin cells.

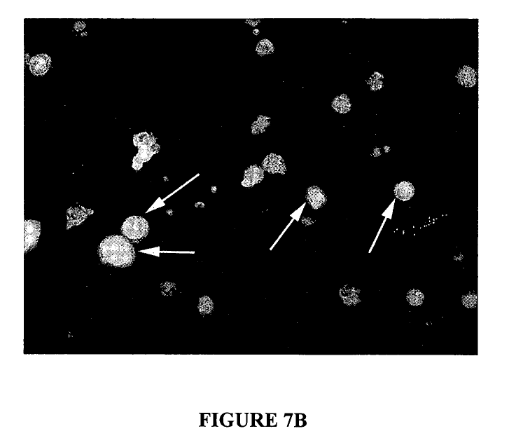

FIG. 8A is a high magnification micrograph of immortalized hCC/TTN/CrePR1 cells (arrows) after dig-insitu hybridization for an antisense riboprobe directed against the neomycin sequence. Cultures of hCC/TTN/CrePR1 cells immortalized with pTTN (loxP) were hybridized with an antisense digoxigenin-labeled riboprobe direct against the neomycin sequence. This neomycin sequence is part of the pTTN (loxP) vector encoded by the immortalized hCCs (after selection with G418 antibiotic). The hCCs are all labeled with the antisense probe suggesting that under the same hybridization conditions, the antisense neo probe recognizes the complementary neo sequence in the cells and will be useful to monitor the disimmortalization completeness with activation of CrePR1 in the cells.

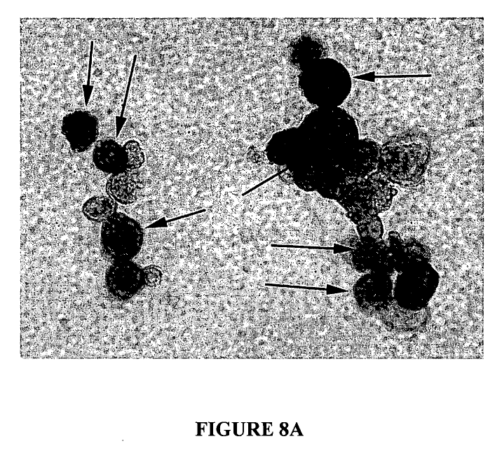

FIG. 8B is a high magnification micrograph of immortalized hCC/TTN/CrePR1 cells (arrows) after dig-insitu hybridization for a sense riboprobe (used at the same concentration and hybridization conditions as with the antisense probe) directed against the neomycin sequence. Cultures of hCC/TTN/CrePR1 cells immortalized with pTTN (loxP) were hybridized with a sense digoxigenin-labeled riboprobe direct against the neomycin sequence. This neomycin sequence is part of the pTTN (loxP) vector encoded by the immortalized hCCs (after selection with G418 antibiotic). The hCCs are all unlabeled with the sense probe suggesting that under the same hybridization conditions, the sense neo probe provides a good negative control in the cells and will be useful to monitor the disimmortalization completeness with activation of CrePR1 in the cells.

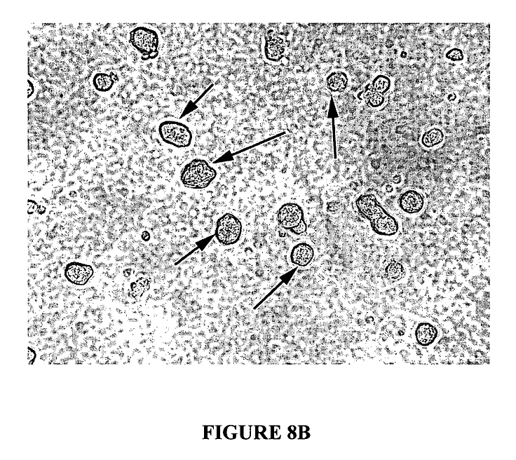

### DETAILED DESCRIPTION OF THE PREFERRED EMBODIMENT(S) OF THE INVENTION

Isolation of Human Fetal Adrenal Medulla Cells

Fetal adrenals from intact whole human embryos (gestational age 7-12 weeks after conception; embryo rump-crown measurements 25-70 mm) were dissected in Mg²⁺—Ca²⁺-free Hank's balanced salt solution (CMF-HBSS) with 1× Gentmicin (Biowhittaker) on ice, mechanically dispersed and then treated with 3 ug/ml Liberase Blendzyme 2/3 (Roche Molecular Biochemicals) at 37 C for 30 min, as described previously (Reference 5). These tissues were obtained from Paule de Viguier Hospital, Toulouse, France, from patients who were electively-aborted with voluntary interruption of pregnancy. These procedures and use of tissue followed a protocol reviewed and approved by the French National Ethics Committee (Reference 3). Dissected tissue was dissociated after trituration through increasingly finer-bore cotton-plugged glass pipettes, and the settled tissue was rinsed ×1 with horse serum (HS; Sigma), filtered (80 um, sterile) into growth media (to remove aggregates), which contained 10% fetal calf serum (FCS) and then concentrated by settling, before replating the top layer of cells. Cells (0.5-1×10⁴), counted by trypan-blue exclusion, were plated on 24-well plate tissue culture plates (Falcon, BDBiosciences) and grown for 7-14 days, at 37 C, CO₂-5%. TC plates were coated with human fibronectin (20 ug/ml; Sigma). Media was changed frequently to remove dead cells, before infection with immortalizing viruses. Balls of nested chromaffin cells were lifted manually and often moved to areas of fibroblasts in the cultures, to disperse the chromaffin cells in culture for infection. Growth media for primary fetal chromaffin cultured cells included: DMEM/Ham=s F12 (D/F; Invitrogen)/10% fetal calf serum (FCS; Sigma); supplemented with insulin (25 ug/ml; Sigma), L-glutamine (L-glu, 2 mM; Eurbio); human beta-fibroblast growth factor (β-FGF, 10 ng/ml; Roche Diagnostic); and 1× Gentamicin(100 ug/ml).

Immortalization of Primary Human Fetal Adrenal Medulla Cells with v-myc or TTN

Cultures were incubated overnight (37EC, 5% CO2) with media (DMEM/Ham's F12 (D/F)/10% FBS/1× pen-strep) with the amphotrophic virus encoding the lox P-flanked sequence for either v-myc (MycTN(LoxP) or SV40 large T antigen (TTN (loxP)), and a positive/negative selection marker (neo resistance for positive selection+HSV TK resistance for negative selection), in the presence of polybrene (0.65-1.25 Fg/ml; Sigma). Amphotrophic MycTN(LoxP) or TTN (loxP) viruses were obtained by transient transfection (Reference 20) of helper-free amphotrophic Phoenix-Ampho packaging cells (ATCC) and titration of resulting infectious particles quantified by qPCR Taqman. Titers were 3.1E5 viral genome/ml (vg/ml) for MycTN(LoxP), and 2.0E5 vg/ml for the TTN(LoxP). Cultures were rinsed with media to remove virus and allowed to rest for 7 days at 37EC, 5% CO2 before treating cultures with the addition of 250 ug/ml of the selection antibiotic G418 (Geneticin; GIBCO) in media, as described above. Media was changed daily for selection, to remove dead cells. When cells were visible after 3-5 days, the FBS % was increased to 20% to increase the rate of proliferation, and the G418 was decreased to 125 ug/ml for maintenance. Cultures were purified by differential plating, to reduce the fibroblast population, before sub-cloning in 100 mm TC dishes (−1,000 cells/plate) to isolate individual colonies of chromaffin cells with subcloning rings. Good survival of chromaffin clones required that some fibroblasts were carried through each subcloning, and each final chromaffin line contains a small number of adherent fibroblasts which must be removed by differential plating (Reference 10) before use.

Differential Plating of Chromaffin Cells

The methods for purification of chromaffin cells to separate them for fibroblasts is a modification of differential plating procedures described by Unsicker and Muller (Reference 21). Briefly, on day 1, cells were lifted from the tissue culture dish with 0.5 mM EDTA/DPBS (sterile, 37 C). The cells were centrifuged 3-5 min at 1000 rpm. The pellet was resuspended in D/F media/10% FBS/pen-strep. Cells were replated in 100 mm non tissue culture plates (cat.#25384-208, VWR; Plainfield, N.J.) with 7-10 ml of media. Cell were incubated 4 hrs at 37 C, for cell lines. Sessile cells (the supernatant that contained cells which did not attach to the plate) were removed into tissue culture flasks (T-25 Blue-top, Falcon) and incubated for 2 hr at 37 C. The supernatant was then moved into T-25 Blue-top flask and further incubated overnight at 37 C.

The next morning, on day 2, the supernatant was removed and saved in 15 ml tube. The flask of attached cells was rinsed briefly with 0.5-1 ml EDTA, 3 mls of media added, and the total solution in the flask used to wash off loose cells, which was then added to the 15 ml tube. The cells were again replated on non-TC plates for 4 hrs, to remove any remaining fibroblasts. Sessile cells in the supernatant were replated to TC flasks (T25 Blue-top, Falcon) for 2 hrs. Media was added to the flask to expand the cells.

Addition of the CrePr1 Construct to Immortalized Human Chromaffin Cells

The p1710(CrePR1) was obtained by inserting, into the p1704 retroviral backbone, the CrePR1 fusion gene (obtained through the fusion of Cre recombinase and the ligand binding domain of the mutant human progesterone receptor HBR891, a kind gift of Dr. Franççois Tronche) upstream of the IRES sequence and a hygromycin-B-phosphotransferase gene downstream. Amphotropic 1710(CrePR1) viruses were obtained by transient transfection (Reference 20) of helper-free amphotrophic Phoenix-Ampho packaging cells (ATCC). The virus titer used for infection of the chromaffin cells was 2.0E5 vg/m. Immortalized chromaffin cultures were incubated overnight (37EC, 5% CO2) with media (D/F/10% FBS/1× pen-strep), containing 0.65-1.25 ug/ml polybrene, with the amphotrophic virus, cells then rinsed with virus-free media, and the cultures rested for 2-3 days before the addition of 400 Fg/ml of the selection antibiotic hygromycin-B (Boehringer-Mannheim). As with initial immortalization, hygromycin concentration was decreased to 125 ug/ml for maintenance, and cultures were purified by differential plating, to reduce the fibroblast population, before sub-cloning in 100 mm TC dishes with subcloning rings to isolate individual colonies of CrePR1-expressing chromaffin cells. Test for the presence of helper virus was performed using the proviral rescue assay (Reference 19) and was found to be negative (see below). After expansion with proliferation, chromaffin cultures were suspended in Cellvation (Celox Labs) freezing medium, according to manufacture's directions, for permanent liquid N₂ cell storage.

Proviral Rescue Assay

Methods used to test for the presence of viral shedding by chromaffin viral-infected cell lines are a modification of those previously published (Reference 19). The indicator cell line used for infection with chromaffin cell conditioned media (CM) was the NIH 3T3 with a nuclear LacZ-expressing integrated provirus (3T3/LZ12) cells. The target cells used for infection with the CM from 3T3/Z12 cells were Rat2 fibroblasts. Briefly, 1 ml of media (DMEM/10% FBS) conditioned for 3 days by confluent human chromaffin cell cultures was made 5 ug/ml with polybrene (Sigma). This CM was added, after filtering through a 0.45 um filter, for 1-3 hr, 37EC, 5% CO2, to 30-50% confluent 3T3LZ12 cells (on 6 cm TC dishes), followed by the addition of 1.5 ml DMEM/10% FBS media (with final polybrene at 2 ug/ml), and the cells allowed to replicate and reach confluency (˜3-4 days). Cells were split 1:10-20, one dish was saved for the assay and polybrene was maintained at 2 ug/ml, until cells reached 50-90% confluence. This step was repeated at least once to increase the sensitivity of the assay. At this point the medium was changed to fresh medium (with maintenance of polybrene) and cells were cultured for an additional two days. The media was harvested, filtered through a 0.45 um filter and polybrene added at a concentration of 8 ug/ml. One ml of CM with polybrene is added to 30% confluent Rat2 fibroblasts, incubated 1-3 hr, 37E C as above, DMEM/10% FBS added in a dilution that made the polybrene concentration 2 ug/ml, and incubation continued for 2-3 times the cell cycle (˜2 days). Cells were then fixed in 2% paraformaldehyde/0.4% glutardaldehyde for 5 min, cells rinsed with ×3 with PBS, and Xgal solution added for 4 hr, 37EC, to stain positive (infected by LacZ provirus) cells blue. The Xgal solution was: in PBS, 4 mM K3Fe(CN)6 (potassium ferricyanide); 4 mM K4Fe(CN)6 .3H2O (potassium ferrocyanide); 2 mM MgCl2. Just before use, X40 Xgal in N,N-dimethyl formamide, to a final concentration of 1 mg/ml, was added.

Nonradioactive Digoxigenin In Situ Hybridization for Neo Expression In Vitro

The methods used are a modification of those previously published (References 11, 8 and 17) for the use of non-radioactive digoxigenin-based RNA/RNA hybridization. Digoxigenin (DIG)-labeled cRNA probes, message complementary (antisense), and noncomplementary (sense), were generated from completely linearized cDNA template using the appropriate T3 and T7 RNA polymerases: antisense, HindIII digestion and T3 RNA polymerase and sense, EcoRI digestion and T7 RNA polymerase utilizing Roche's DIG RNA Labeling materials and methods. Three riboprobe plasmids were tested, all inserted into BlueScript vector (pBSII-SK+; Stratagene). A BstBI/EcoRI 438 bp fragment of the neo sequence; a NcoI/EcoRI 636 bp fragment of the neo sequence; and a PstI 532 bp fragment of the neo sequence. After mRNA transcription, riboprobes were quantified with a known amount of control DIG-labeled RNA and utilizing Roche's DIG Quantification Teststrips and DIG Wash & Block Buffer Set, following manufacturer's recommendations.

Methods for nonradioactive in situ hybridization for neo riboprobes were adapted from those previously described (Reference 13), with minor modifications (Reference 18) utilizing the DIG Nucleic Acid Detection Kit from Roche, following manufacturer=s recommendations. Briefly, cell cultures of chromaffin cells, purified with differential plating as above, were grown on substrate-plated RNAse-free glass slides (Enzo Life Sci., Farmindale, N.Y.; Cat.# 31802-100), without phenol-red (in D/F/10% FBS/pen-strep). Cells were washed with PBS (pH 7.4) at 37 C, then fixed at room temperature(RT) for 30 min in a solution of 4% (w/v) formaldehyde, 5% (v/v) acetic acid, and 0.9% (w/v) NaCl. The fixed cells were then rinsed ×3 with PBS at room temperature and insitu procedures were usually begun immediately after fixation. Before in situ hybridization, the fixed cells were dehydrated as follows: incubating successively in 70%, 90%, and 100% ethanol; washed in 100% xylene to remove residual lipids; and rehydrated by incubating successively in 100%, 90%, and 70% ethanol, and finally, incubated in PBS. Then, the fixed cells were incubated at 37 C with 0.1% (w/v) pepsin in 0.1 N HCl, to increase permeability to macromolecular reagents, followed by a wash with PBS for 5 min; post-fix with 1% formaldehyde for 10 min; and washed again with PBS. Prehybridization utilizing 150 l of the hybridization solution on each slide (containing 60% deionized formamide, 300 mM NaCl, 30 mM sodium citrate, 10 mM EDTA, 25 mM NaH2PO4 (pH 7.4), 5% dextran sulfate, and 250 ng/μl sheared salmon sperm DNA) was performed for 2 hrs, at 28-36 C, in a humidified oven (slides covered with Hybri-slip; Sigma) before the solution was replaced with new hybridization solution (containing 50-200 ng/ml of riboprobe in hybridization solution) and hybridization continued overnight at 28-36 C (depending on probe size), 18 hrs in a humidified oven. Stringency washes for optimal specific hybridization signal was as follows: after hybridization, coverslips were removed by shaking the slides at room temperature in a solution of 60% formamide, 300 mM NaCl, and 30 mM sodium citrate. The slides were then washed as follows: 3× at room temperature, followed by 1× at 37 C. The slides were washed 1×5 min in PBS, followed by 5 min at room temperature with 100 mM Tris-HCl (pH 7.5), 150 mM NaCl. then the slides were incubated for 30 min at room temperature with blocking buffer [100 mM Tris-HCl (pH 7.5), 150 mM NaCl; saturated with blocking reagent]. Binding of DIG-labeled RNA hybrids with an anti-DIG Fab fragment (1:200 dilution) (DIG Nucleic Acid Detection Kit) conjugated to alkaline phosphatase (120 min at RT), followed by washing the slides as follows: 2×5 min at room temperature with 100 mM Tris-HCl (pH 7.5), 150 mM NaCl; then the slides were covered with detection buffer containing 0.18 mg/ml 5-bromo-4-chloro-3-indolylphosphate (BCIP), 0.34 mg/ml NBT, and 240 μg/ml levamisole and incubate for up to 16 h at room temperature. Controls, consisting of sections subjected to the complete in situ hybridization procedure, but with no probe added, or hybridized with sense probe for neo exhibited no specific hybridization signal.

Excision of v-myc (or TTN) from Immortalized Human Chromaffin Cells

Immortalized chromaffin cells, expressing v-myc or SV40 Tag and CrePR1, were proliferated to near confluence at 37EC, in DMEM/Ham's F12 (DF) growth media/10% FBS/125 ug/ml G418/125 ug/ml hygromycin as described previously above (or HL-1 media, if we do this) on collagen-coated TC plates, before switching for 14 days to the dissimmortalization media (without G418 or hygromycin). Separate cultures of cells were left untreated or incubated for the first 7 days in the presence of 1 uM RU486, with 40 ug/ml gancyclovir added the last 7 days of treatment, or treated for 14 days with RU486 alone at 37EC. (If necessary, cultures were differentially plated to remove fibroblasts after excision and settled overnight on substrate-coated slides before in situ). Without in situ, differentiation of disimmortalized chromaffin cells was continued in disimmortalization media with the addition of 5 ug/ml dexamethasone (Reference 10) and 500 uM tetrahydrobiopterin (BH4), the co-factor for catecholamine synthesis (Reference 1).

V-myc or large T antigen expression was examined in untreated, RU486-treated, and RU486/gancylovir-treated surviving chromaffin cells with non-radioactive in situ hybridization for expression of the neo sequence, encoded with v-myc and SV40 Tag in the immortalizing vectors. Riboprobes incorporating Dig-UTP were transcribed from the Bluescript expression vector (T3/T7) with 438-636 bp inserts (3) subcloned from neo. (Portion of TK-neo positive/negative selection sequence in the v-myc plasmid (the hybrid resistance gene obtained through the fusion of the genes for the herpes simplex virus thymidine kinase (HSV TK) and the bacterial neomycin phosphotransferase (neo) (from the plasmid pTNFUS69 kindly donated by R. Kucherlapati.

Immunohistochemistry

For characterization of the chromaffin cell lines using immunocytochemistry, cells were proliferated to near confluence at 37 C. Cultures were differentially plated to remove fibroblasts and settled overnight on 8-well plastic slides. Cells were fixed with 4% paraformaldehyde in 0.1M phosphate buffer at pH 7.4, for 20 minutes, rinsed with phosphate buffered saline (PBS), and nonspecific background blocked with the preimmune serum, for a few hours at the room temperature. Cells were reacted with primary antibodies for 18-48 hours at 4 C, followed by several rinses and incubation in secondary fluorescent antibodies (1-2 hours at room temperature). Secondary antibody reporters were: goat anti-mouse or goat anti-rabbit IgG Alexa green or Alexa red from Molecular Probes (Eugene, Oreg.). After reactions were completed, slides were cover slipped using anti-fade mounting medium, Vectashield from Vector Laboratories (Burlingame, Calif.). Cell preparations were scanned with Zeiss inverted confocal microscope (Axiovert 100M), using argon LASOSLGK 7812 ML4/LGN 7812:458, 477, 488, 514 nm, 30 nW laser, furnished with software package LSM 510. Images were collected at the 488 nm wavelength and scanned at 200 dpi into TIF format and images collected in Adobe Photoshop.

Tyrosine hydroxylase (TH)

Briefly, before primary antibody incubation, cells were incubated with 5% normal goat serum (NGS) in PBS/0.4% TX, for a few hours at the room temperature. Cells were then incubated for overnight at 4 C with rabbit polyclonal antibody to TH (Chemicon), diluted 1:300 in PBS/0.4% TX/5% NGS, rinsed, followed by anti-rabbit IgG-fluorescein conjugate (Alexa green, Molecular Probes), diluted 1:100 in PBS, and incubated 1 hr at room temperature.

Dopa-β-hydroxylase(DβH)

Some cultured cells were also stained for DβH expression to assess whether disimmortalized chromaffin cells continued to express DβH. Antibody staining for DβH in the cells is a modification of methods described previously (Reference 2). The anti-DBH polyclonal antibody, raised in rabbit (1:300; Incstar; Stillwater, Minn.) was incubated with TX-permeabilized (0.4%/PBS) cells overnight at 4 C, followed by a anti-rabbit Alexa green secondary reporter.

Phenylethanolamine-N-methyltransferase (PNMT)

Some cultured cells were co-labeled for Tag and also stained for PNMT expression to assess whether the chromaffin cells continued to express PNMT and were capable of synthesizing epinephrine after excision of tsTag. In a separate experiment that used double-labeling, the Tag marker was also used in these cultures to examine whether PNMT-positive cells contained Tag-ir. Antibody staining for PNMT in the cells is a modification of methods described previously (Reference 4). The anti-PNMT polyclonal antibody, raised in rabbit (1:300; Incstar; Stillwater, Minn.) was incubated under the same conditions as described above for polyclonal antibodies.

Met-enkephalin

Briefly, before primary antibody incubation, cells were incubated with 5% normal goat serum (NGS) in PBS/0.4% TX, for a few hours at the room temperature. Cells were then incubated for overnight at 4 C with rabbit polyclonal antibody to MET-ENK (Immunostar Cat # 20065) 1/100 dilution in blocking buffer in PBS/0.4% TX/5% NGS, rinsed, followed by anti-rabbit IgG-fluorescein conjugate (Alexa green, Molecular Probes), diluted 1:100 in PBS, and incubated 1 hr at room temperature.

Chromagranin

Briefly, before primary antibody incubation, cells were incubated with 5% normal goat serum (NGS) in PBS/0.4% TX, for a few hours at the room temperature. Cells were then incubated for overnight at 4 C with rabbit polyclonal antibody to chromagranin (Immunostar 20085) 1/200 dilution in PBS/0.4% TX/5% NGS, rinsed, followed by anti-rabbit IgG-fluorescein conjugate (Alexa green, Molecular Probes), diluted 1:100 in PBS, and incubated 1 hr at room temperature.

Chemicals

Primary antiserum against the indicated antigens were obtained from the following sources: dopamine beta-hydroxylase (DβH), Chemicon (Temecula, Calif.); phenylethanolamine-N-methyltransferase (PNMT), Incstar (Stillwater, Minn.); and tyrosine hydroxylase (TH), Chemicon (Temecula, Calif.). Vectastain Elite ABC kits (anti-mouse and anti-rabbit IgG) from Vector Laboratories (Burlingame, Calif.); BCIP/NBT substrate, Proteinase K, DIG RNA Labeling Kit, DIG Quantification Teststrips, DIG Nucleic Acid Detection Kit, High Pure Plasmid Isolation Kit, and DIG Wash & Block Buffer Set, which contained the materials for in situ hybridization with non-radioactive digoxigenin detection of riboprobes were from Roche Molecular Biochemicals (Mannhein, Germany); all restriction enzymes used from New England BioLabs (Beverly, Mass.); RNA polymerases were from Promega (Madison, Wis.). Hybri-slips were obtained from Sigma Chemical (St. Louis, Mo.). Slides used for in situ were obtained from Enzo Life Sciience (Farmindale, N.Y.). Ham's F12 media (D/F, 1:1, vol/vol) and Geneticin (G418) were obtained from GIBCO (Grand Island, N.Y.); Cellvation was from Celox Labs. Gentamicin sulfate was obtained from Biowhittaker, Walkersville, Md.; L-glutamine from Eurbio, LesVilles, France. All other powdered media, attachment factors and chemicals for cell culture were purchased from Sigma Chemical (St. Louis, Mo.). Fetal bovine serum (FBS) was obtained from Hyclone (Logan, Utah). The RU486 was supplied by Biomol Research Lab, Inc. (Plymouth Meeting, Mass), and the antibiotic gancyclovir was obtained from Roche Laboratories (Nutley, N.J.). Cellstripper, Mediatech, Inc. (Herndon, Va.), a proprietary non-enzymatic cell dissociation solution was used to lift chromaffin cells after differentiation for antibody staining. Vectashield mounting medium (with DAPI) was obtained from Vector Laboratories (Burlingame, Calif.). Non-tissue cultures plates for differential plating were obtained from VWR (Plainfield, N.J.).

While this invention has been described as having preferred sequences, ranges, steps, materials, structures, features, and/or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention and of the limits of the appended claims.

The following references, and those cited or discussed herein, are all hereby incorporated herein in their entirety by reference.

### REFERENCES

1. Abou-Donia, M. M., Wilson, S. P., Zimmerman, T. P., Nichol, C. A., Viveros, O. H. Regulation of guanosine triphosphate cyclohydrolase and tetrahydrobiopterin levels and the role of the cofactor in tyrosine hydroxylation in primary cultures of adrenomedullary chromaffin cells. J Neurochem, 46, 1190-1199 (1986).
2. Aunis, D., Hesketh, J., Devilliers, G. Immunohistochemical and immunocytochemical localization of myosin, chromogranin A and dopamine-β-hydroxylase in nerve cells in culture and adrenal glands. J Neurocytol, 9, 255-274 (1980).
3. Bachoud-Levi, A. C., Remy, P., Nguyen, J. P., Brugieres, P., Lefaucheur, J. P., Bourdet, C., Baudic, S., Gaura, V., Maison, P., Peschanski, M. Motor and cognitive improvements in patients with Huntington's disease after neural transplantation. Lancet, 356, 1975-1979 (2000).
4. Cahill, A. L., Eertmoed, L., Mangoura, D., Perlman, R. Differential regulation of phenylethanolamine-N-methyltransferase expression in two distinct subpopulations of bovine chromaffin cells. J. Neurochem., 67, 1217-1224 (1996).
5. Cairns L, Crotta S, Minuzzo M, Ricciardi-Castagnoli P, Pozzi L, Ottolenghi S: Immortalization of neuro-endocrine cells from adrenal tumors arising in SV40 T-transgenic mice. Oncogene 14:3093-3098 (1997).
6. Czech K A, Sagen J: Update on cellular transplantation into the CNS as a novel therapy for chronic pain. Prog. Neurobiol. 46:507-529 (1995).
7. Duplan H, Bes J C, Tafani M, Sallerin B, Sagen J, Ohayon E, Lazorthes Y, Tkaczuk J: Adrenal medullary explants as an efficient tool for pain control: adhesive biomolecular components are involved in graft function ex vivo. Exp Neurol, 163:331-347(2000).
8. Eaton, M. J., Blits, B., Ruitenberg, M. J., Verhaagen, J., Oudega, M. Amelioration of chronic neuropathic pain by adeno-associated viral (AAV) vector-mediated overexpression of BDNF in the rat spinal cord. Gene Ther, 9, 1387-1395 (2002a).
9. Eaton M J: Emerging cell and molecular strategies for the study and treatment of painful peripheral neuropathies. J Peripher Nerv Sys 5:59-74 (2000).
10. Eaton, M. J., Frydel, B., Lopez, T., Nie, X., Huang, J., Sagen, J. Generation and initial characterization of conditionally immortalized chromaffin cells. J. Cell Biochem., 79, 38-57 (1999).
11. Eaton, M. J., Gudehithlu, K. P., Quach, T., Silvia, C. P., Hadjiconstantinou, M., Neff, N. H. Distribution of aromatic L-amino acid decarboxylase mRNA in mouse brain by in situ hybridization histology. J. Comp. Neurol., 337, 640-654 (1993).
12. Eaton, M. J., Herman, J. P., Jullien, N., Lopez, T., Martinez, M., Huang, J. Immortalized chromaffin cells disimmortalized with Cre/lox site-directed recombination for use in cell therapy for pain. Exp Neurol, 175, 49-60 (2002b).
13. Giger, R. J., Wolfer, D. P., De Wit, G. M., Verhaagen, J. Anatomy of rat semaphorin III/collapsin-1 mRNA expression and relationship to developing nerve tracts during neuroembryogenesis. J Comp Nerol, 375, 378-392 (1996).
14. Lazorthes Y, Sagen J, Sallerin B, Tkaczuk J, Duplan H, Sol J C, Tafani M, Bes J C: Human chromaffin cell graft into the CSF for cancer pain management: a prospective phase 11 clinical study. Pain 87:19-32 (2000).
15. Muench, M. O., Ratcliffe, J. V., Nakanishi, M., Ishimoto, H., Jaffe, R. B. Isolation of definitive zone and chromaffin cells based upon expression of CD56 (neural cell adhesion molecule) in the human fetal adrenal gland. J Clin Endocrinol Metab, 88, 3921-3930 (2003).
16. Paillard F: Reversible cell immortalization with the cre-lox system. Hum. Gene Ther. 10:1597-1598 (1999).
17. Pal, R., Eaton, M. J., Islam, S., Hake-Frendscho, M., Kumar, K. N., Michaelis, E. K. Immunocytochemical and in situ hybridization studies of the expression and distributional of three subunits of a complex with N-methyl-D-aspartate receptor-like properties. Neurosci., 94, 1291-1311 (1999).
18. Pasterkamp, R. J., Giger, R. J., Ruitenberg, M. J., Holtmaat, A., De Wit, J., de Winter, F., Verhaagen, J. Expression of the gene encoding the chemorepellent semaphorin III is induced in the fibroblast component of neural scar tissue formed following injuries of adult but not neonatal CNS. Mol Cell Neurosci, 13, 143-166 (1999).
19. Pear, W. S. III Transduction of genes using retrovirus vectors. In: Ausabel, F. M., Brent, R., Kingston, R. E., Moore, D. D., Seigman, J. G., Smith, J. A. & Struhl, K. (eds),. Current protocols in molecular biology. John Wiley and Sons, New York (1995).
20. Pear, W. S., Nolan, G. P., Scott, M. L., Baltimore, D. Production of high-titer helper-free retroviruses by transient transfection. Proc. Nat. Acad. Sci., U.S.A., 90, 8392-8396 (1993).
21. Unsicker, K. & Muller, T. H. Purification of bovine adrenal chromaffin cells by differential plating. J. Neurosci. Meth., 4, 227-241 (1981).
22. Wang H, Sagen J: Attenuation of pain-related hyperventilation in adjuvant arthritic rats with adrenal medullary transplants in the spinal subarachnoid space. Pain 63:313-3201 (1995).
23. Duplan et al, Generation of conditionally immortalized cell lines genetically modified to over-express opioid peptides for chronic pain control, (Abstract) Exp Neurol, 175 (2): 433 (2002).
24. Wang X-T, Eaton M J. Sagen J. Generation of conditionally immortalized serotonergic pineal cells for transplantation in chronic pain. Soc Neurosci, Abstract 23: 1455 (1997).
25. Kim P, Seasholtz A F, Sagen J. Generation of opioid-producing PC12 cells for transplantation in chronic pain. Amer Soc Neural Transpl, Abstract 3:34 (1996).
26. Kim P H, Wang X-T, Seasholtz A, Sagen J, Unnerstall J R. A met-enkephalin producing PC12 cell line as an alternative donor source for neural transplants in the reduction of chronic pain. Soc Neurosci, Abstract 22: 1021 (1996).

### U.S. PATENTS/APPLICATIONS

27. Anderson David J. Immortalization and disimmortalization of cells. U.S. Pat. No. 5,629,159, issued May 13, 1997.
28. Gage Fred H. et al. Method of grafting genetically modified cells to treat defects, disease or damage to the CNS. U.S. Pat. No. 5,082,670, issued Jan. 21, 1992.
29. Sagen J, Pappas G D, Perlow M J, Pollard H B: Inducing analgesia by implantation of cells releasing neuroactive substances. U.S. Pat. No. 4,753,635, issued Jun. 28, 1988.
30. Sagen J, Pappas G D, Sortwell C E: Alleviation of depression by the transplantation of monoamine-containing cells in the CNS. U.S. Pat. No. 4,980,174, issued Dec. 25, 1990.
31. Sagen J, Pappas G D: Implantable cells that alleviate chronic pain in humans. U.S. Pat. No. 5,656,267, issued Aug. 12, 1997.
32. Sagen J: A method to prevent opiate tolerance. U.S. Pat. No. 5,762,925, issued Jun. 9, 1998. Reversing opioid tolerance by cellular implantation. U.S. Pat. No. 5,730,974, issued, Mar. 24, 1998.
33. Sagen J: Reversing excitotoxic damage by cellular implantation. U.S. Pat. No. 5,935,606, issued Aug. 10, 1999.

What is claimed is:

1. An immortalized mammalian cell that expresses chromaffin cell phenotype.

2. The cell of claim 1, wherein the cell comprises a human cell.

3. An immortalized mammalian cell that expresses chromaffin cell genotype.

4. The cell of claim 3, wherein the cell comprises a human cell.

5. A cloned mammalian cell that expresses chromaffin cell phenotype.

6. The cell of claim 5, wherein the cell comprises a human cell.

7. A cloned mammalian cell that expresses chromaffin cell genotype.

8. The cell of claim 7, wherein the cell comprises a human cell.

9. A disimmortalizable mammalian cell that expresses chromaffin cell phenotype.

10. The cell of claim 9, wherein the cell comprises a human cell.

11. A disimmortalizable mammalian cell that expresses chromaffin cell genotype.

12. The cell of claim 11, wherein the cell comprises a human cell.

13. A cloned mammalian cell line that releases a chromaffin-related antinociceptive agent.

14. The cell line of claim 13, wherein the cell line comprises a human cell line.

15. A disimmortalizable mammalian chromaffin cell line.

16. A disimmortalizable human chromaffin cell line.

17. A disimmortalizable mammalian chromaffin cell line for transplant into a region of the central nervous system of a subject to induce analgesia.

18. The cell line of claim 17, wherein the region comprises the subarachnoid space near the spinal cord.

19. A disimmortalizable human chromaffin cell line for transplant into a region of the central nervous system of a subject to induce analgesia.

20. The cell line of claim 19, wherein the region comprises the subarachnoid space near the spinal cord.
